(19) United States
(12) Patent Application Publication
(10) Pub. No.: US 2005/0248255 A1
(43) Pub. Date: Nov. 10, 2005

(54) Light source structure with deodorization and bacteria-repelling functions and method for manufacturing the same

(76) Inventor: Ying-Chen Chen, Hsin Chu Hsien (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/839,348

(22) Filed: May 6, 2004

(51) Int. Cl. H01J 1/62

(52) U.S. Cl. 313/493

(57) ABSTRACT

A light source structure with deodorization and bacteria-repelling functions and method for manufacturing the same is described. A specific surface of a light source is roughened. The light source has a light source tube and at least a photocatalyst colloid film coated on an outer rough surface of the light source tube. The thickness of the photocatalyst colloid film is less than the roughness of the outer surface of the light source tube. The light source structure of the present invention can increase the effective area of the photocatalyst colloid film and enhance the effectiveness of deodorization and bacteria-repelling.

### BACKGROUND OF THE INVENTION

1. Field of the Invention

The present invention relates to a light source structure with deodorization and bacteria-repelling functions and a method for manufacturing the same, and especially to a light source structure with a photocatalyst colloid film coated on a rough surface thereof.

2. Description of Prior Art

Reference is made to FIG. 1 and FIG. 2 at the same time, which illustrate a conventional light source 10a. The light source 10a includes a light source body 11a and a photocatalyst material 20a coated on a smooth outer surface of the light source body 11a. When the photocatalyst material 20a is irradiated with light emitted from the light source 10a, a photocatalytic reaction takes place, which is capable of deodorization and repelling bacteria, by decomposing most of the organic substance and part of the inorganic material in the air. In other words, an airborne contaminant is decomposed when adheres to photocatalyst material 20a and the photo-catalytic reaction takes place. When the airflow is high speed or contains a high density of contaminants, it is obvious that a photocatalyst material film with large area is desired to absorb and decompose the airborne contaminant, and a limited area of photocatalyst material film is not sufficient to decompose all the airborne contaminant. FIG. 2A is a 3000 times enlargement of the outer surface of the light source 10a before the photocatalyst material 20a is coated thereon. FIG. 2B is a 1000 times enlargement of the outer surface of the light source 10a with the photocatalyst material 20a coated thereon. FIG. 2A and FIG. 2B show that the outer surface of the light source 10a is always smooth.

Further reference is made to Taiwan Patent No. 562235, which discloses a light source with a colloid film coated on an inner surface thereof, with visible light fluorescent powder and ultraviolet fluorescent powder mixed and distributed in the colloid film. A photocatalyst colloid film is coated on an outer surface of the transparent light source body, the ultraviolet fluorescent powder is stimulated and radiates more ultraviolet light, the ultraviolet light passes through the light source body and irradiates the photocatalyst colloid film. In this way, the ability to decompose airborne contaminants on the photocatalyst material is enhanced.

However, the prior art as described has some disadvantages. Both the surfaces of the light source, with or without the photocatalyst colloid film coated thereon, are smooth. Thus, the effective area of the photocatalyst colloid film does not increase after coating, and the insufficient effective area may lead to poor performance. When the conventional structure encounters a density of contaminants in the air, a high proportion of airborne contaminants may not be decomposed completely after prolonged use. When the conventional structure encounters high-speed airflow, part of the airborne contaminants may still remain in the airflow and not be absorbed and decomposed.

Thus, it is necessary to provide a new light source structure with deodorization and bacteria-repelling functions and a method for manufacturing the same.

### SUMMARY OF THE INVENTION

An object to the present invention is to provide a light source structure with deodorization and bacteria-repelling functions and a method for manufacturing the same by roughening a specific surface of the light source structure for increasing both the surface area of the light source and the effective surface of a photocatalyst colloid film coated thereon.

To achieve the above object, a light source structure with deodorization and bacteria-repelling function in accordance with a preferred embodiment of the present invention comprises a light source tube and at least a photocatalyst colloid film coated on an outer rough surface of the light source body. The photocatalyst colloid film is thinner than the roughness of the outer surface of the light source tube.

The method for manufacturing the light source structure with deodorization and bacteria-repelling function comprises processes described as follows. A transparent light source body is provided and a specific surface thereof is roughened. A photocatalyst material is coated on the specific surface of the light source body. The photocatalyst material is solidified for forming a photocatalyst colloid film on the specific surface of the light source body.

In this way, the light source structure of the present invention can increase the effective area of the photocatalyst colloid film and enhance the performance of deodorization and bacteria repelling.

### BRIEF DESCRIPTION OF THE DRAWINGS

Other objects, advantages, and novel features of the present invention will become more apparent from the following detailed description when taken in conjunction with the accompanying drawings, in which:

FIG. 1 is a perspective view of a conventional light source;

FIG. 2 is a cross-sectional view of a conventional light source;

FIG. 2A shows a 3000 times enlargement of an outer surface of the conventional light source, before coating with a photocatalyst colloid film thereon;

FIG. 2B shows a 1000 times enlargement of an outer surface of the conventional light source, with a photocatalyst colloid film coated thereon;

FIG. 3 is a perspective view of a light source structure according to the present invention;

FIG. 4 is a cross-sectional view of the light source structure shown in FIG. 3;

FIG. 4A is a partial, enlarged view of the light source structure shown in FIG. 4;

FIG. 5 is a partial, enlarged view of another embodiment of FIG. 4;

FIG. 6 illustrates one application of the light source structure of the present invention in which the light source structure is circular in shape;

FIG. 7 illustrates another application of the light source structure of the present invention in which the light source structure is U-shaped;

FIG. 8 is a schematic view of a light source structure of the present invention with a photocatalyst colloid film just coated on an upper half partial surface thereof; and

FIG. 9 illustrates an embodiment of a light source structure of the present invention in which a photocatalyst colloid film is strip shapes with an interval between adjacent strip shaped photocatalyst colloid films.

FIG. 10 is a schematic view of an assembly of a light source structure of the present invention and a transparent semi-cylinder cover;

FIG. 11 is a schematic view of an assembly of a light source structure of the present invention and a transparent cylinder cover;

FIGS. 11A to 11F are schematic views illustrating round-shaped or square-shaped transparent covers of the present invention;

FIG. 12 is a schematic view of a light source structure of the present invention when applied to a lamp;

FIG. 13 is a schematic view of a light source structure of the present invention when applied to an air cleaner;

FIG. 14 is a schematic view of a light source structure of the present invention when applied to a storage container;

FIG. 15 is a schematic view of a light source structure of the present invention when applied to a desk lamp;

FIG. 16 is a schematic view of a light source structure of the present invention when applied to a backlight module;

FIG. 17 is a schematic view of a light source structure of the present invention when applied to an electrical heater;

FIG. 18 is a 1000 times enlargement of a rough surface of a light source of the present invention after roughening;

FIG. 19 is a 1000 times enlargement of a rough surface of a light source of the present invention with a photocatalyst colloid film coated thereon;

FIG. 20 shows a comparison of the performance for CH3COOC4H9/4L catalyzing and decomposing between a light source with smooth surface and a light source with rough surface, in which both light sources surface are coated with photocatalyst colloid film and emit light with a wavelength of 365 nm to irradiate the photocatalyst colloid film; and

FIG. 21 shows a comparison of the performance for CH3COOC4H9/4 L catalyzing and decomposing between a light source with smooth surface and a light source with rough surface, in which both light sources surface are coated with photocatalyst colloid film and emit light of a wavelength of 543 nm to irradiate the photocatalyst colloid film.

### DETAILED DESCRIPTION OF THE INVENTION

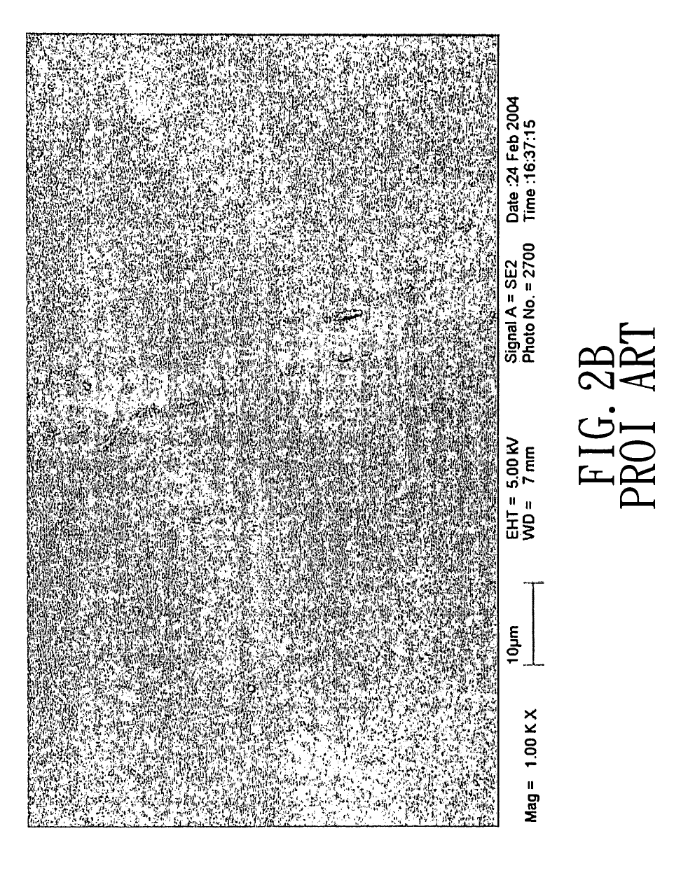

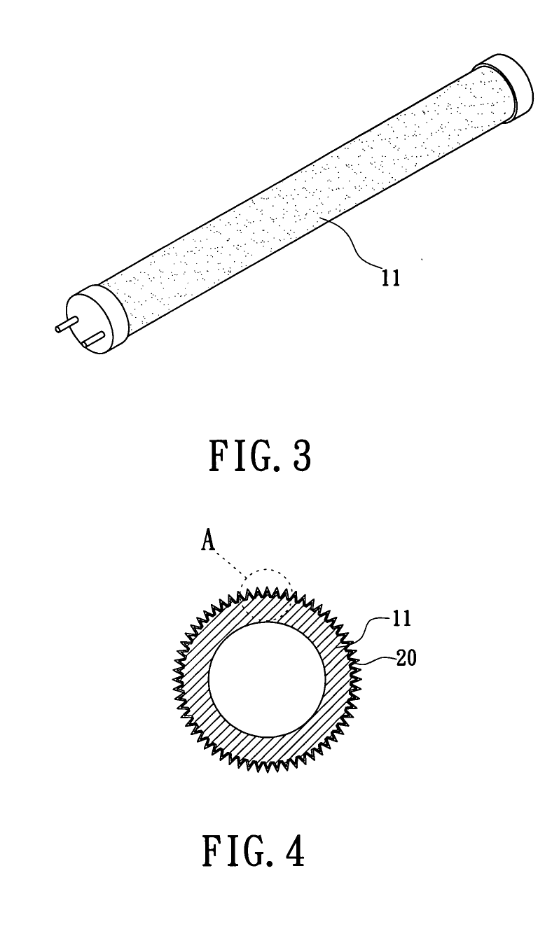

Reference is made to FIG. 3 and FIG. 4. A light source structure with deodorization and bacteria repelling functions and a method for manufacturing the same are provided. The light source structure comprises a transparent tube used as light source body 11 and a photocatalyst colloid film 20. The light source body 11 has a rough outer surface. The photocatalyst colloid film 20 is coated on the rough outer surface of the light source body 11. The thickness of the photocatalyst colloid film 20 is less than the roughness of the rough surface of the light source body 11. In this way, the light source of present invention has larger effective area for absorption and decomposition of an airborne contaminant than a conventional light source as stated before.

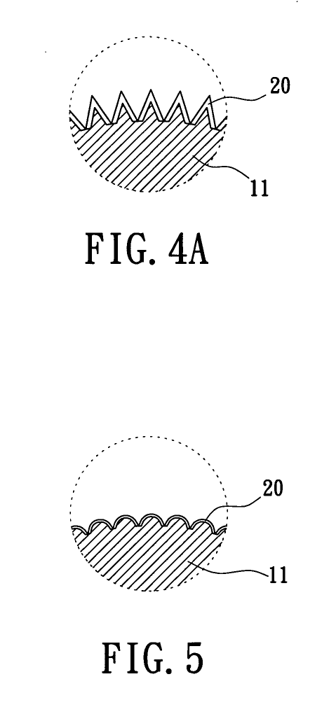

Referring to FIG. 3 to FIG. 5, the rough surface of the light source body 11 can be formed by physical or chemical methods and processed one or more times. The chemical method to form rough surface uses certain materials that can react chemically with the light source body 11, which is normally made of glass. For example, hydrofluoric acid can react with glass, is suitable for etching, and after etching and scrubbing with hydrofluoric acid, the outer surface of the light source body 11 become rough. The physical method can be sandblasting or whetting; either the physical method or the chemical method used to roughen the outer surface of the light source body 11 can achieve the object of increasing the area of outer surface of the light source body 11. As shown in FIG. 4A and FIG. 5, the distance between adjacent protruding portions of the rough outer surface is 0.5 μm to 10 μm, and the thickness of the photocatalyst colloid film is 0.05 μm to 0.2 μm, so the photocatalyst colloid film 20 can be coated closely on the rough surface. The protruding portion of the rough surface can be acuate or round in shape. The light source body 11 is transparent and light can pass therethrough. The light rays scatter when they pass through the rough surface of the light source body 11 and the photocatalyst colloid film 20 coated thereon, which softens the lighting and reduces glare.

The photocatalyst colloid film 20 can be coated on the rough surface of the light source body 11 in the following processes. First, photocatalyst material is coated on the rough surface of the light source body 11 by print coating, immersion coating, spray coating, drench coating or brushing. The photocatalyst material is then solidified by drying to form the photocatalyst colloid film 20. Because the photocatalyst colloid film 20 is closely coated on the rough surface of the light source body 11, the effective area of the photocatalyst colloid film 20 whereon the photo-catalyst reaction takes place is increased. Hence, the performance of deodorization and bacterial repelling is improved. Further, catalyst contained in the photocatalyst colloid film 20 is TiO₂, SnO₂, WO₃, Fe₂O₃, SrTiO₃, ZnO, or a combination thereof. The photocatalyst colloid film 20 can further contain heavy metal, such as Au, Pt, Pd, or Ag, or a salt of a transition metal, such as Mo, Ce, Nb, V, or Cr, which can further improve catalytic performance of the photocatalyst colloid film 20.

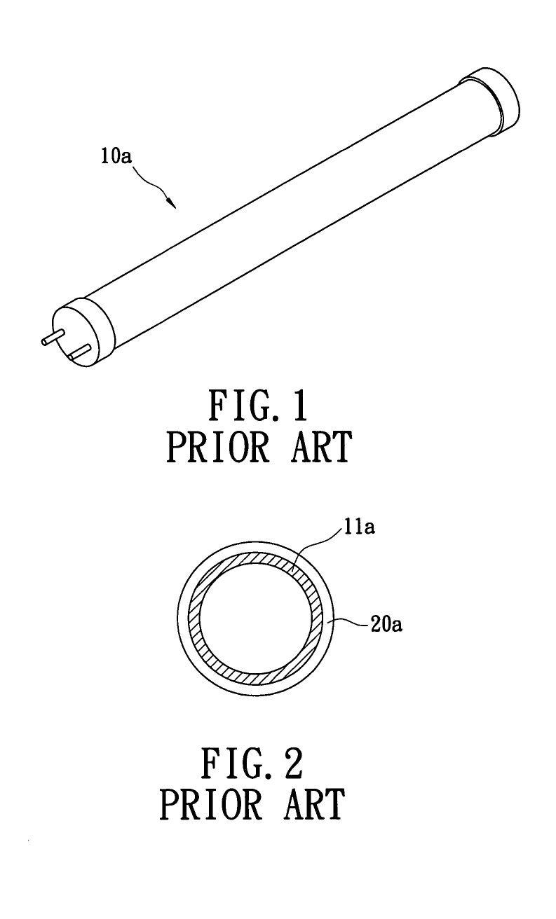

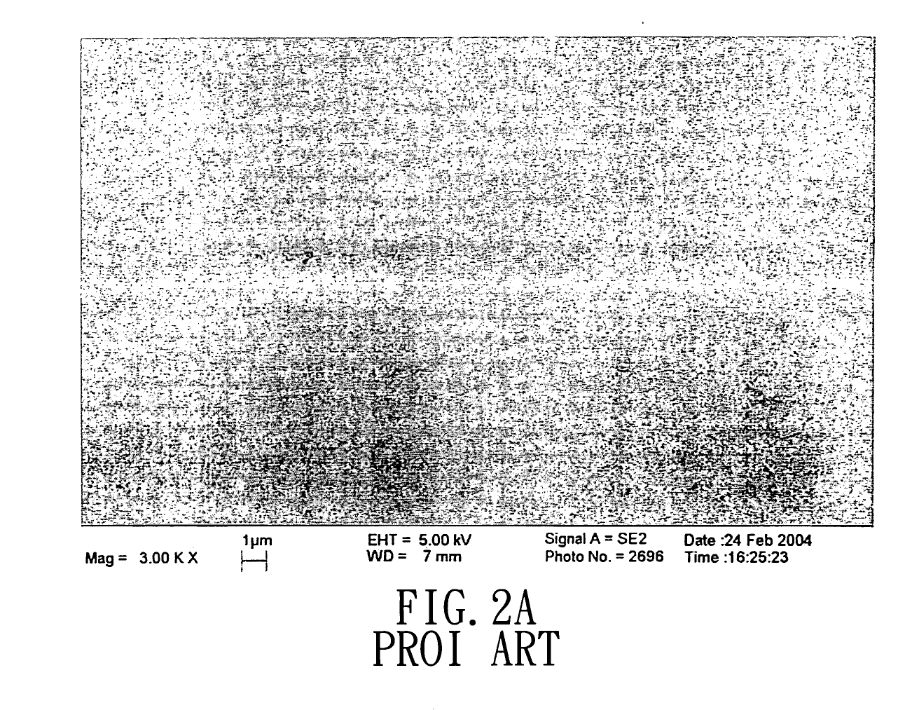

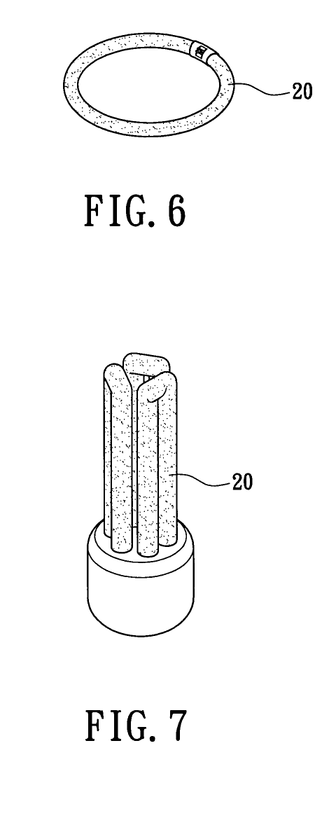

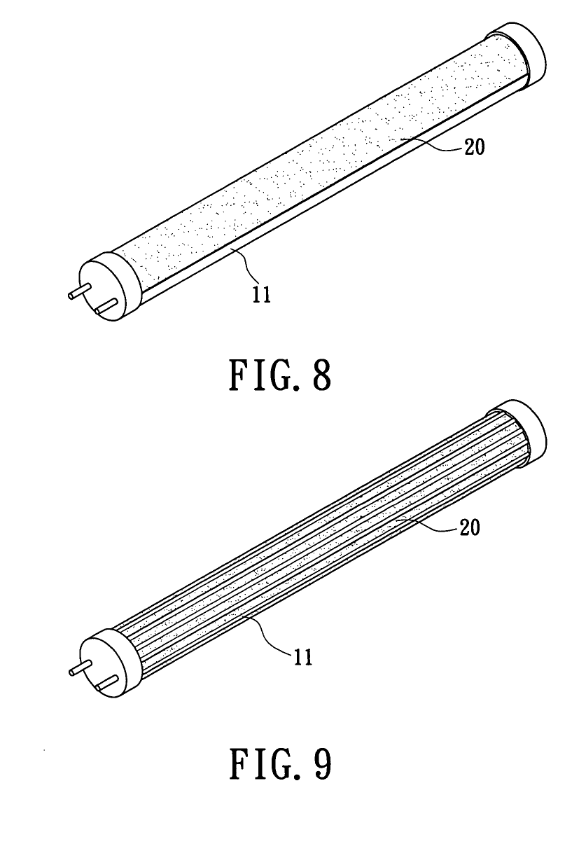

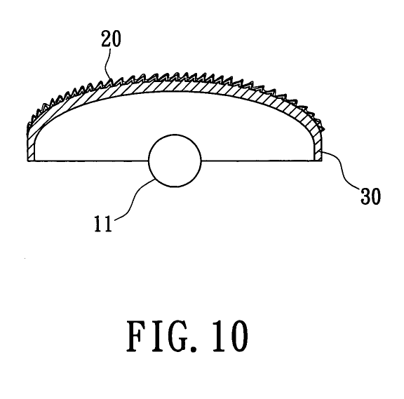

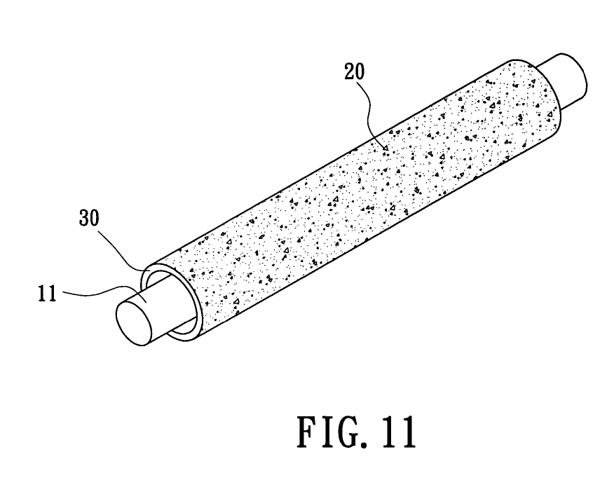

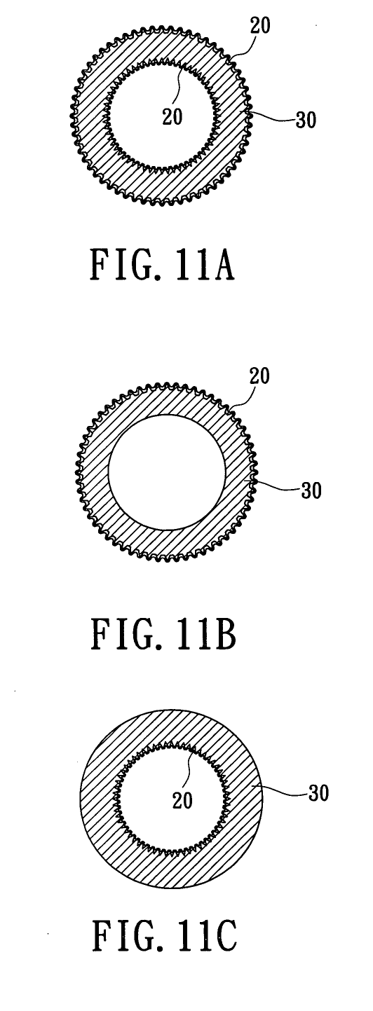

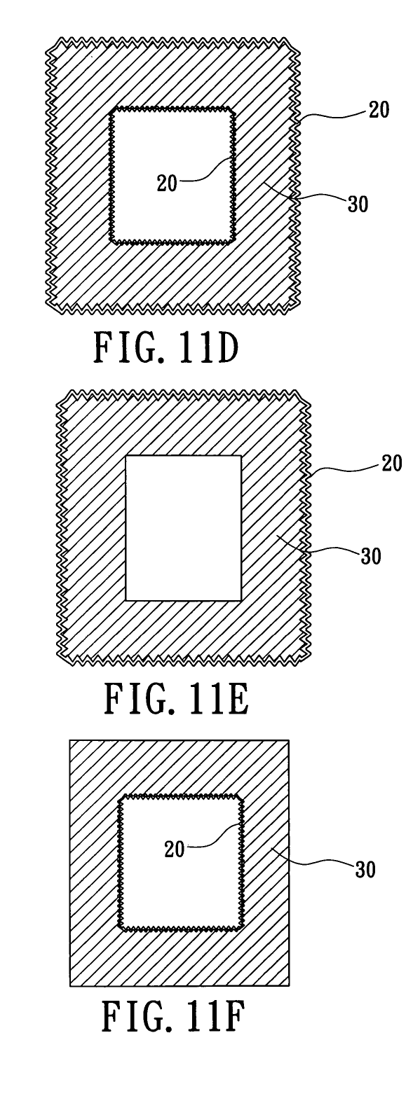

Reference is made to FIG. 6 and FIG. 7; the light source body can be a cylinder, a circular tube, a twisted tube, a cold cathode tube or a U-shape. The photocatalyst colloid film 22 coated on the surface of the light source body 11 can be in strips, with an interval between the strips of photocatalyst colloid film. The photocatalyst colloid film 20 can just be coated on part of the cover, e.g. the upper half partial surface of the light source body, as shown in FIG. 8, or distributed on all surfaces of the light source body 11, as shown in FIG. 9. FIG. 10 shows another embodiment of the present invention, which comprises a transparent cover 30 adjacent to the light source body 11. The transparent cover 30 has a rough surface, on which a photocatalyst colloid film 20 is coated. The transparent cover 30 can be a half-surrounding shape or a complete-surrounding shape, and the transparent cover 30 is formed surrounding the light source body 11, as shown in FIG. 1. FIG. 11A-FIG. 11F show several embodiments of the present invention, in which the cross-section of the transparent cover can be a circle or a square, either the inner or outer surface of the transparent cover 30 is rough, and the photocatalyst colloid film 20 is coated on the rough surface of the transparent cover 30. Because the thickness of the photocatalyst colloid film 20 is less than the roughness of a specific rough surface of the transparent cover 30, the photocatalyst colloid film 20 can be coated closely on the rough surface the transparent cover 30.

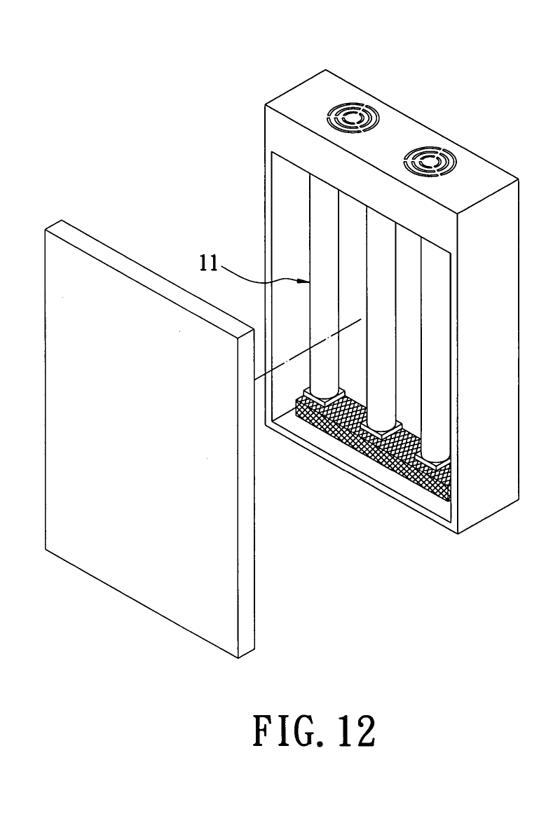

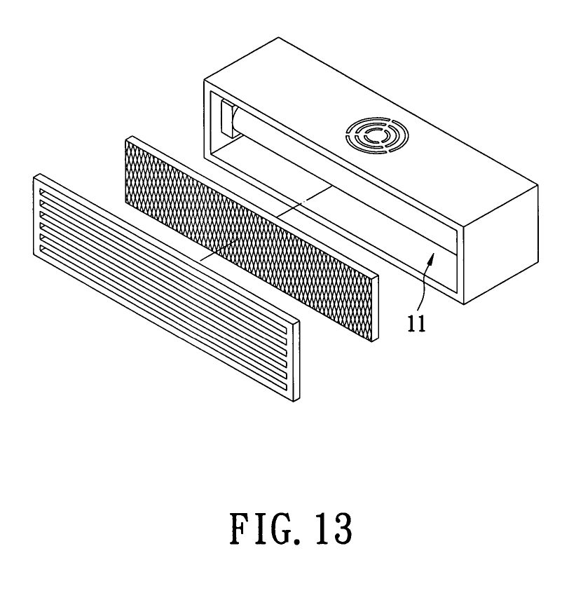

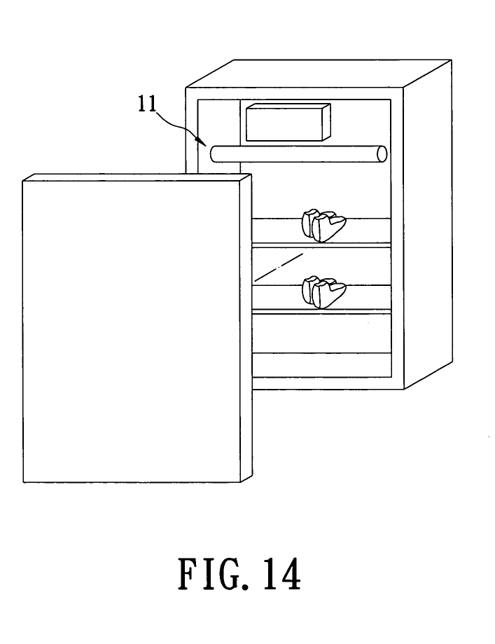

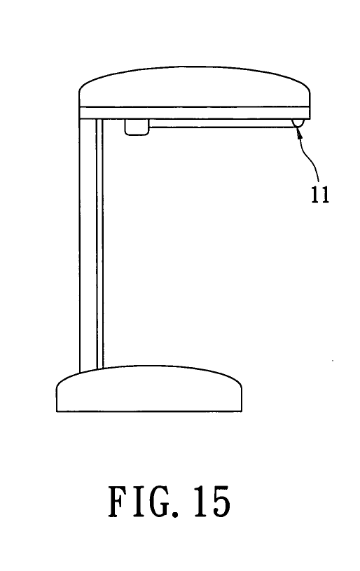

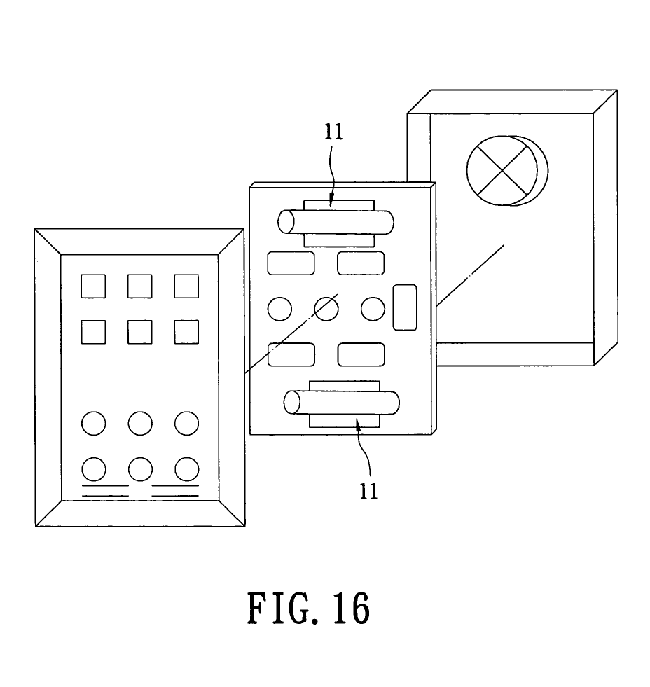

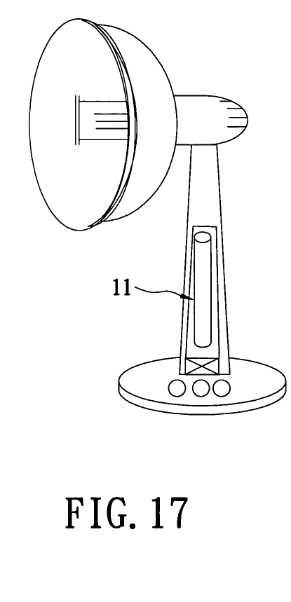

The light source structure with deodorization and bacteria-repelling function of the present invention can be applied to a lamp as shown in FIG. 12, applied to a air cleaner, as shown in FIG. 13, applied to a storage container, as shown in FIG. 14, applied to a reading lamp, as shown in FIG. 15, applied to a backlight module as shown in FIG. 16, and applied to a electrical heater as shown in FIG. 17.

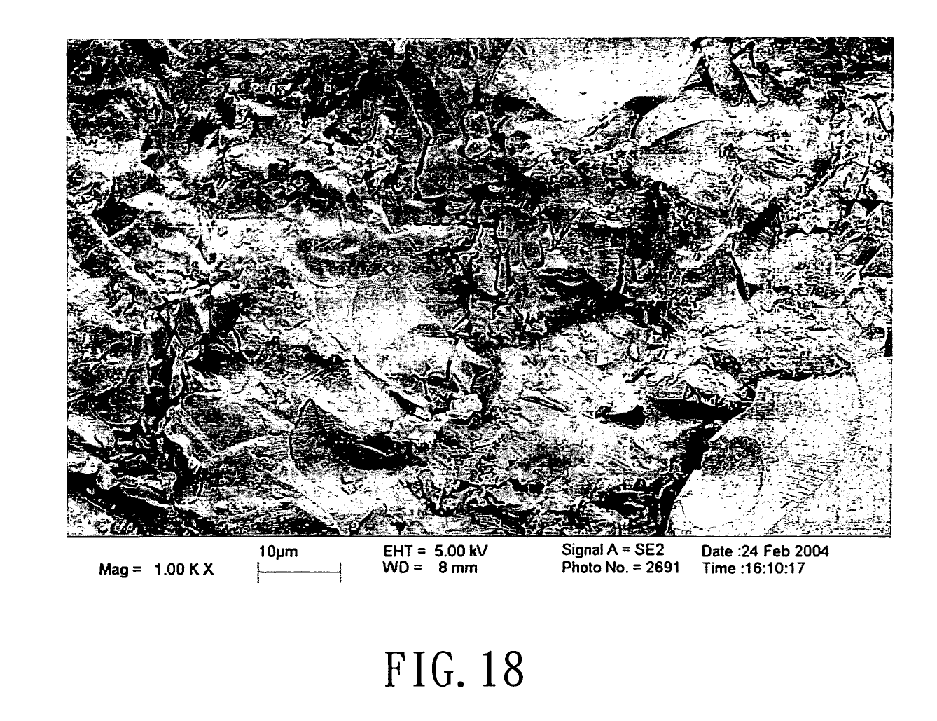

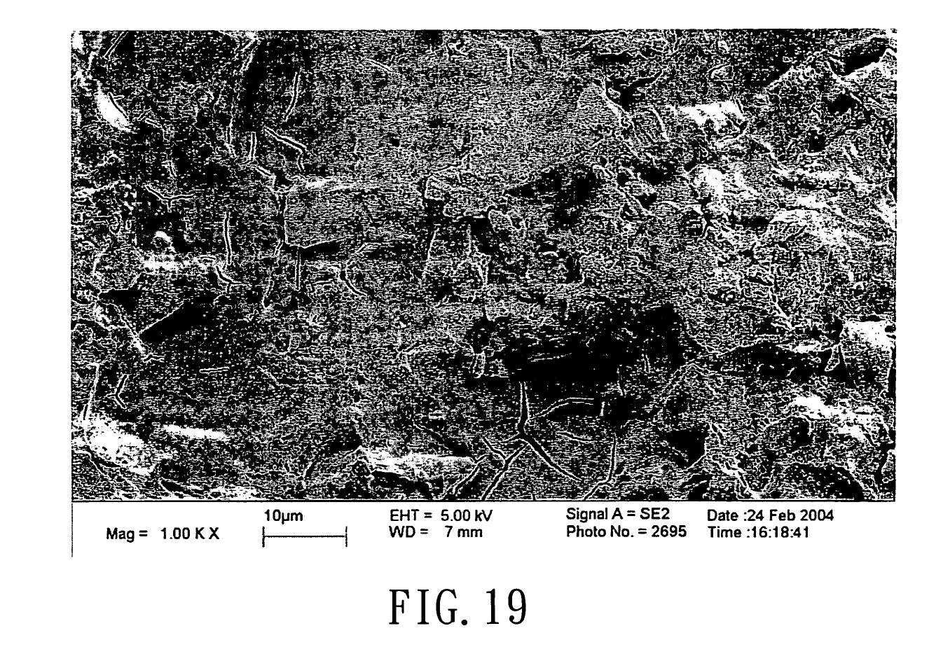

Reference is made to FIG. 18, which shows an enlargement of the rough surface of the light source body 11 of the present invention after roughening. Reference is made to FIG. 19 as well, which shows an enlargement of the rough surface of the light source body 11 of the present invention with the photocatalyst colloid film 20 coated thereon. For a certain light source body, surface area of a light source body with rough surface is undoubtedly larger than that of a light source body with a smooth surface, so the effective area of the photocatalyst colloid film 20 coated on the rough surface of the light source body 11 is larger than that of the photocatalyst colloid film 20 coated on a smooth surface.

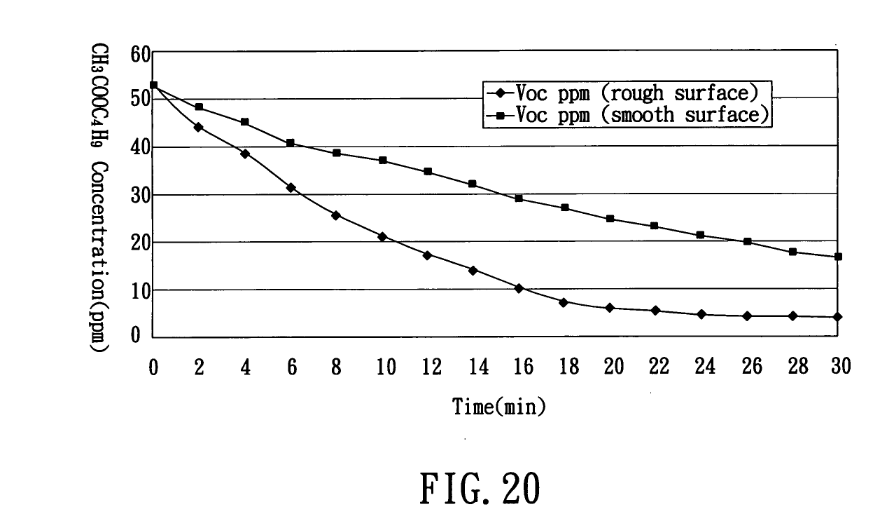

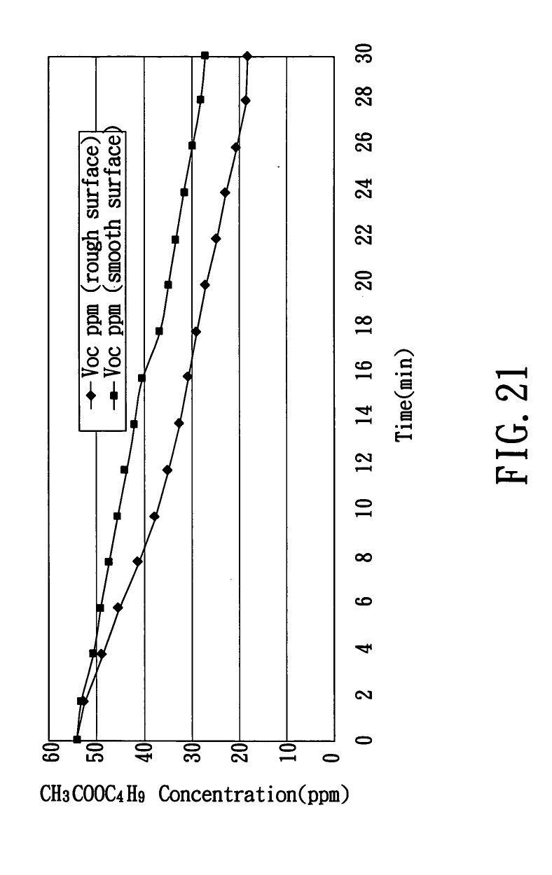

FIG. 20 is performance comparison between two light source bodies 11 with rough surface and with smooth surface. Powers of both the light source bodies 11 are 8 w. Each light source body 11 emits light to irradiate the photocatalyst colloid film 20 coated thereon for catalyzing and decomposing CH₃COOC₄H₉/4 L. The wavelength of the light emitted from each light source body 11 is 365 nm. FIG. 20 shows that the performance of the light source body 11 with the rough surface is better than that of the light source body 11 with the smooth surface. Referring to FIG. 21, when the wavelength of the light emitted from each light source body 11 is changed to 543 nm, the performance of the light source body 11 with the rough surface is better than that of the light source body 11 with the smooth surface, just as in the previous case.

The light source structure with deodorization and bacteria-repelling function and the method for manufacturing the same of the present invention can effectively increase area of the light source body 11 and the effective area of the photocatalyst colloid film 20, which improves performance of the light source, and promotes the capability of absorption and decomposing airborne contaminant for the light source structure of the present invention.

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

1. A light source structure with deodorization and bacteria-repelling functions, comprising:
a light source body including a rough outer surface; and
a photocatalyst colloid film applied on the rough outer surface of the light source body, wherein a thickness of the photocatalyst is less than a surface roughness of the outer surface of the light source body.

2. The light source structure with deodorization and bacteria-repelling functions as described in claim 1, wherein the photocatalyst colloid film comprises a photocatalyst material selected from a group consisting of TiO₂, SnO₂, WO₃, Fe₂O₃, SrTiO₃, ZnO and a combination thereof.

3. The light source structure with deodorization and bacteria-repelling functions as described in claim 1, wherein the photocatalyst colloid film further comprises a photocatalyst material selected from a group consisting of heavy metals, transition metal salts, and a combination thereof.

4. The light source structure with deodorization and bacteria-repelling functions as described in claim 1, wherein the photocatalyst colloid film further comprises a photocatalyst material selected from a group consisting of Au, Pt, Pd, Ag, Mo, Ce, Nb, V and Cr.

5. The light source structure with deodorization and bacteria-repelling functions as described in claim 1, wherein the photocatalyst colloid film is applied on a partial area of the rough outer surface of the light source body.

6. The light source structure with deodorization and bacteria-repelling functions as described in claim 1, wherein the photocatalyst colloid film coated on the rough outer surface of the light source body is strip shaped and adjacent strips of photocatalyst colloid film are separated by an interval.

7. The light source structure with deodorization and bacteria-repelling functions as described in claim 1, further comprising a transparent cover adjacent to the light source body, the transparent cover having a photocatalyst colloid film formed on one rough outer surface thereof.

8. The light source structure with deodorization and bacteria-repelling functions as described in claim 7, wherein the transparent cover has a rough outer surface.

9. The light source structure with deodorization and bacteria-repelling functions as described in claim 7, wherein the transparent cover has a rough inner surface.

10. The light source structure with deodorization and bacteria-repelling functions as described in claim 7, wherein the transparent cover includes outer and inner surfaces.

11. The light source structure with deodorization and bacteria-repelling functions as described in claim 7, wherein the transparent cover is a transparent half-cover.

12. The light source structure with deodorization and bacteria-repelling functions as described in claim 7, wherein the transparent cover is an orbicular shaped transparent cover.

13. The light source structure with deodorization and bacteria-repelling functions as described in claim 1, wherein a protruding portion of the rough outer surface is acuate in shape.

14. The light source structure with deodorization and bacteria-repelling functions as described in claim 1, wherein a protruding portion of the rough outer surface is round.

15. The light source structure with deodorization and bacteria-repelling functions as described in claim 1, wherein a distance between adjacent protruding portions on the rough surface is about 0.5 μm to 10 μm.

16. A method for manufacturing the light source structure with deodorization and bacteria-repelling functions, the method comprising:
providing a light source body;
roughening a specific surface of the light tube;
coating a photocatalyst material on the specific surface of the light source body; and
solidifying the photocatalyst material to form a photocatalyst colloid film on the specific surface of the light source body.

17. A method for manufacturing the light source structure with deodorization and bacteria-repelling functions as described in claim 16, wherein the method to roughen the specific surface comprises mechanical roughening, chemical etching, or a combination thereof.

18. A method for manufacturing the light source structure with deodorization and bacteria-repelling functions as described in claim 16, wherein the photocatalyst material is solidified to form the photocatalyst colloid film on the specific surface by drying.

19. A method for manufacturing the light source structure with deodorization and bacteria-repelling functions as described in claim 16, wherein the specific surface is roughened by chemical etching.

20. A method for manufacturing the light source structure with deodorization and bacteria-repelling functions as described in claim 16, wherein the photocatalyst material is adhered to the specific surface by print coating, immersion coating, spray coating, drench coating, brushing, or a combination thereof.
